(19) United States
(12) Patent Application Publication
(10) Pub. No.: US 2014/0221468 A1
(43) Pub. Date: Aug. 7, 2014

(54) Compounds that Modulate Store Operated Calcium Channels

(71) Applicants: Chan Young Park, Stanford, CA (US); Amir M. Sadaghiani, Stanford, CA (US); Ricardo Ei Dolmetsch, Stanford, CA (US)

(72) Inventors: Chan Young Park, Stanford, CA (US); Amir M. Sadaghiani, Stanford, CA (US); Ricardo Ei Dolmetsch, Stanford, CA (US)

(73) Assignee: The Board of Trustees of the Leland Stanford Junior University, Palo Alto, CA (US)

(21) Appl. No.: 13/990,961

(22) PCT Filed: Dec. 9, 2011
(86) PCT No.: PCT/US11/64234

§ 371 Date: Feb. 5, 2014

Related U.S. Application Data
(60) Provisional application No. 61/459,322, filed on Dec. 9, 2010.

(51) Int. Cl. C07D 493/06; C07D 407/04; C07D 311/38; C07C 63/66; C07D 321/10; C07D 493/04

(52) U.S. Cl. CPC C07D 493/06; C07D 321/10; C07D 493/04; C07D 311/38; C07C 63/66; C07D 407/04
USPC 514/450; 549/349; 549/354; 549/387; 549/403; 549/435; 560/129; 435/375; 514/456; 514/455; 514/510; 514/470; 514/464

(57) ABSTRACT

Store operated calcium channel modulating compounds, and methods for using the same, are provided. These compounds and methods find use in a variety of applications in which modulation of store operated calcium channels is desired.

### GOVERNMENT RIGHTS

This invention was made with Government support under grants R01 NS048564 and R01 GM45374 awarded by the National Institutes of Health. The Government has certain rights in this invention.

### BACKGROUND OF THE INVENTION

Calcium has an important role in many cellular functions such as the transduction of signals into and within cells. Processes that depend on calcium are involved in cellular responses to growth factors, neurotransmitters, hormones and other signal molecules. Intracellular calcium concentrations are tightly controlled by a variety of pumps and channels. As calcium is the most abundant second messenger in cell signaling, such tight control is necessary to produce specific calcium mediated responses.

Store Operated Calcium Entry, which involves Calcium Release-Activated Calcium (CRAC) channels and their currents (I_{CRAC}), is one of many ways of regulating intracellular calcium levels. Store-operated calcium channels (SOCs) comprise the major receptor-activated calcium entry pathway in non-excitable mammalian cells and play important roles in the control of gene expression, cell differentiation, secretion and calcium homeostasis. In their native environment, SOCs are activated by the stimulation of phospholipase C (PLC)-coupled receptors that generate inositol 1,4,5-trisphosphate (IP3) and release Ca²⁺ from the endoplasmic reticulum (ER). The defining feature of SOCs is that they are activated by the reduction of calcium in the ER rather than by receptor-associated signaling molecules, such as G proteins, PLC or IP3.

The CRAC channel is an extensively characterized store-operated channel whose activation is a step function of Ca²⁺ in the ER. CRAC channels play an essential role in the initiation of the adaptive immune response in T-lymphocytes as well as mast cells and are central to immunity in humans, as a single loss-of-function mutation in the CRAC channel leads to severe combined immunodeficiency. CRAC channels provide the pathway for Ca²⁺ entry triggered by antigen recognition or allergens, and are required for T-cell activation and mast cell degranulation.

Two major components of SOC have been identified. STIM, a type one single trans-membrane protein resides mainly at the endoplasmic reticulum (ER) membrane and Orai, a four trans-membrane protein is localized at the plasma membrane. Upon binding of an extra cellular ligand to a G-protein coupled receptor (GPCR), cytosolic IP3 is produced that in turn binds to IP3 receptors on the ER surface. The depletion of ER calcium due to the release of calcium through IP₃ receptors is then detected by the EF calcium binding hand of STIM. STIM is then thought to change conformation and oligomerize and expose an important Orai binding domain called CRAC Activating Domain (CAD). Direct binding of CAD to the intracellular domains of Orai, mainly N and C-termini, results in oligomerization of STIM and Orai and activation of SOC. These clusters of STIM and Orai form a series of puncta on the plasma membrane that are only visible after depletion of ER and activation of SOC. During this stage, the influx of calcium can be visualized and measured using colorimetric dyes and electrophysiological techniques.

Orai1 accumulates in overlying regions of the plasma membrane (PM) in register with STIM1, culminating in the local entry of Ca²⁺ through CRAC channels. A recent study shows that STIM1 oligomerization is the key event that triggers the redistribution of STIM1 and Orai1, translating changes in the Ca²⁺ concentration of the ER into graded activation of the CRAC channel.

STIM1 forms puncta in response to store depletion even when expressed in the nominal absence of Orai1, suggesting that its initial target may be independent of Orai1. In contrast, Orai1 only forms puncta in store-depleted cells when co-expressed with STIM1, suggesting that it becomes trapped at ER-PM junctions by binding to STIM1 or an associated protein. Several parts of the cytosolic domain of STIM1, including the C-terminal polybasic domain, an ERM-like domain, and a serine-proline-rich domain, have been implicated in the activation of Orai1.

The molecular mechanism by which STIM1 activates the CRAC channel has also been studied. A widely considered ‘diffusible messenger’ model posits that STIM1 oligomerization promotes the synthesis of a ‘Ca²⁺ influx factor’ (CIF), which is delivered locally at ER-PM junctions to stimulate iPLA2β to produce lysolipids that activate ICRAC. An alternative ‘conformational coupling’ hypothesis proposes that STIM1 binds physically to the CRAC channel or to an associated protein to activate Ca²⁺ entry. One recent study has proposed that STIM1 links Orai1 dimers to form active tetrameric channels, while another study concludes that Orai1 is a tetramer at rest, suggesting instead that STIM1 activates Orai1 by an allosteric mechanism.

STIM and Orai are expressed fairly ubiquitously throughout most different tissue types. Immune cells, especially T lymphocytes have been a cell type used to study SOC. SOC is the major way of calcium entry in T lymphocytes and plays the main role in their activation. Once SOC is activated the increase of intracellular calcium concentration results in dephosphorylation of Nuclear Factor for Activated T cells (NFAT) by calcineurin, and its delocalization to the nucleus. Diacylglycerol (DAG), a side product of IP3 production from phosphatidylinisitol 4,5-bisphosphate (PIP2), activates AP1 through the protein kinase C (PKC) pathway. AP1 and NFAT will then act in tandem to turn on many immune related cytokine genes including IL2 and IL4 that are necessary for the immune system functionality.

Human mutations in Orai1 and STIM1 cause Severe Combined Immunodeficiency (SCID) syndrome and non-progressive muscular dysplasia due to the immune cells lacking the ability to produce SOC. Mutations that cause irregularities in calcium homeostasis by altering the SOC activity have been linked to neurological pathologies such as Huntington and Alzheimer's disease. Additionally, Genetic manipulations of SOC reduce the damage from ischemia and thrombus formation in mice. STIM and Orai are of interest as targets for discovery of drugs for inflammatory diseases such as rheumatoid arthritis and asthma along with many other conditions such as ischemia and graft versus host.

The immunosuppressants tacrolimus and cyclosporin, target and inhibit the activity of calcineurin, and also interact with a wide variety of other drugs and substances. Calcineurin, a down-stream component of the SOC pathway, has been linked to many brain receptor chemicals including NMDA and shown to have interaction with many other proteins like AKAP5. Inhibition of calcineurin, especially using such non-specific reagents like tacrolumous and cyclosporin could be responsible for over a dozen adverse side effects of currently available immunosuppressants.

Small molecule inhibitors of SOC that target STIM and Orai proteins specifically are of interest to modulate calcium signaling in a variety of systems including the immune system as immunosuppressants

The adaptive immune response can be activated or inhibited by altering calcium influx in T-lymphocytes. An inhibition of the immune response could clinically be used to suppress organ rejection during transplantation and to normalize immune response in cases of immunological disorders such as autoimmune diseases, inflammation or hypersensitivity. An activation of the immune response would be desirable in cases of immunodeficiencies, where immune function is compromised. It would be beneficial for either case to develop agents that modulate calcium influx in T-lymphocytes.

Store operated calcium channels formed by the STIM and Orai family of proteins play central roles in the function of a variety of cells including T and B lymphocytes, mast cells, platelets and skin cells. Modulators of these channels are of interest for treating autoimmune diseases, graft vs host disease, thrombosis, cancer and allergies.

Related publications

Related publications include Li et al. (2007). J Biol Chem 282, 29448-29456; Liou, et al (2007) Proc Natl Acad Sci USA 104, 9301-9306; Liou et al (2005) Curr Biol 15, 1235-1241; Huang et al (2006). Nat Cell Biol 8, 1003-1010; Zhang et al. (2005) Nature 437, 902-905.

Mullins et al., Proc. National Academy of Sciences (2009) “Stim1 and Calmodulin interact with Orai1 to induce calcium-dependent inactivation of Crac channels.”

Park et al., Cell 136:876-90 (2009) “STIM1 Clusters and Activates CRAC channels via Direct Binding of a Cytosolic Domain to Orai1.”

### SUMMARY OF THE INVENTION

Store operated calcium channel modulating compounds are provided, and methods of using the same, e.g., to modulate intracellular calcium. Compounds of interest include those set forth in Structures I-XIII herein. Compounds of the invention find use in binding to members of the Stim and Orai family of proteins, modulating store operated calcium influx, and preventing expression of proinflammatory genes. These compounds and methods find use in a variety of applications in which modulation of store operated calcium channels is desired.

In some embodiments of the invention, pharmaceutical compositions are provided, which comprise an effective dose of a compound identified herein, which dose is effective in modulating intracellular calcium concentrations; and which composition may further comprise a physiologically acceptable excipient.

The above summary is not intended to include all features and aspects of the present invention nor does it imply that the invention must include all features and aspects discussed in this summary.

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 illustrates the chemical binding screen against minimal functional domains of Stim and Orai: (A) Expressed regions of Stim, CAD, and intracellular domains of Orai are represented in red; (B) SDS PAGE of the GST fused proteins; (C) Cartoon depiction of Small Molecule Microarray (SMM) binding screen; (D) Histogram of replicate averaged hits for GST-CAD (right) and GST fused intracellular domains of Orai (left); (E) X-Y scatter plots of normalized Z scores for the binding of the chemicals (Z-scores A-C=replicates; red=background binding; blue=true test compounds; cyan=hits).

FIG. 2 illustrates the identification and validation of inhibitors of SOC by NFAT-luc assays: (A) NFAT-luc screen results of 51 compounds (top) and Renilla-luc control of cell viability (bottom); (B)-(E) NFAT-luc assay results for selected compounds and analogs.

FIG. 3 illustrates the inhibition SOC by Fura2-AM calcium imaging in HEK293 cells: (A), (C) Cells treated with 50 μM compounds 1 minute before treatment with thapsigargin; (B), (D) Bar graph of the slopes of calcium influx from fura2-AM imaging; (E) Post SOC activation treatment of the compounds (DMSO (black), LaCl at 1 uM (red), AnCoA4 at 50 uM (orange), and BiochaninA at 50 uM (green) at minute 510 of the calcium imaging).

FIG. 4 illustrates the down-regulation of T and B lymphocyte activating genes by RT PCR after treatment of AnCoA4: (A) Log scaled fold downregulation of 84 genes by qRT PCR (green=down two fold, red=up 2 fold); (B) Semi qRT PCR of IL2 expression in jurkat T cells; (C) Heat map representation of the expression of 84 genes; (D) Bar graph representation of the significantly down-regulated genes.

### DEFINITIONS

The term “EF hand”, as used herein, relates to a helix-loop-helix structural domain found in a large family of calcium-binding proteins. It consists of two alpha helices positioned roughly perpendicular to one another and linked by a short loop region (usually about 12 amino acids) that usually binds calcium ions. The motif takes its name from traditional nomenclature used in describing the protein parvalbumin, which contains three such motifs and is probably involved in muscle relaxation via its calcium-binding activity. EF hands also appear in each structural domain of the signaling protein calmodulin and in the muscle protein troponin-C.

A “STIM polypeptide” or “STIM protein” is a polypeptide or protein that is the same as, a splice-variant of, or homologous to a naturally occurring STIM polypeptide or protein, or that is derived from such a polypeptide or protein (e.g., through cloning, recombination, mutation, or the like). The polypeptide can be full length or can be a fragment of a full length protein. A STIM fragment typically includes at least 10 contiguous amino acids corresponding to a native STIM protein. The polypeptide or protein can be naturally occurring or recombinant, and can be unpurified, purified, or isolated, and can exist, e.g., in vitro, in vivo, or in situ. The STIM polypeptide is a member of a highly conserved gene family that includes two known homologs (STIM1, STIM2) in the human STIM family of proteins. Any of a variety of STIM polypeptides or proteins and coding nucleic acids can be used in the present invention.

The term “CAD”, as used herein, is an acronym for a 107-residue CRAC activation domain within the C-terminus of STIM1. The CAD sequence is (SEQ ID NO:1): YAPEALQKWL QLTHEVEVQY YNIKKQNAEK QLLVAKEGAEK IKKKRNTLFG TFHVAHSSSL DDVDHKILTA KQALSEVTAA LRERLHRWQQ IEILCGFQIV NNPGIH.

The term “SOCs”, as used herein, denominates store-operated Ca²⁺ channels. The term “SOCE”, as used herein, is an acronym for store-operated Ca²⁺ entry. The term “ICRAC”, as used herein, denominates Ca²⁺ release-activated Ca²⁺ current. The terms “intracellular calcium” and “intracellular Ca²⁺”, as used herein, generally refer to “cytosolic calcium” and “cytosolic Ca²⁺” in a cell. The terms “intracellular stores”, “Ca²⁺ stores and “calcium stores”, as used herein, generally refer to calcium that is sequestered in the endoplasmic reticulum or other organelles in a cell.

An “Orai polypeptide” or “Orai protein” is a polypeptide or protein that is the same as, a splice-variant of, or homologous to a naturally occurring Orai polypeptide or protein, or that is derived from such a polypeptide or protein (e.g., through cloning, recombination, mutation, or the like). The polypeptide can be full length or can be a fragment of a full length protein. An Orai fragment typically includes at least 10 contiguous amino acids corresponding to a native Orai protein. The polypeptide or protein can be naturally occurring or recombinant, and can be unpurified, purified, or isolated, and can exist, e.g., in vitro, in vivo, or in situ. The Orai polypeptide is a member of a highly conserved gene family that includes three known homologs (Orai 1, Orai2 and Orai 3) in the human Orai family of proteins. Any of a variety of Orai polypeptides or proteins and coding nucleic acids can be used in the present invention.

Inhibitory Orai polypeptides (see co-pending international application US2010/025529, herein specifically incorporated by reference) include: (SEQ ID NO:2) Orai NT (1-91) MHPEPAPPPS RSSPELPPSG GSTTSGSRRS RRRSGDGEPP GAPPPPPSAV TYPDWIGQSY SEVMSLNEHS MQALSWRKLY LSRAKLKASS R; (SEQ ID NO:3) Orai NT (48-91) SAVTYPDWIG QSYSEVMSLN EHSMQALSWR KLYLSRAKLK ASSR; (SEQ ID NO:4) Orai NT (64-91) MSLNEHSMQA LSWRKLYLS RAKLKASSR; (SEQ ID NO:5) Orai II-III (142-177) TCILPNIEAV SNVHNLNSVK ESPHERMHRH IELAWA; (SEQ ID NO:6) Orai CT (256-301) HFYRSLVSHK TDRQFQELNE LAEFARLQDQ LDHRGDHPLT PGSHYA. When such peptides are brought into contact with a CRAC channel they will inhibit the opening of the channel and ion influx there through, for example by inhibiting endogenous Stim proteins. Such inhibition can have the effect of inhibiting a T cell mediated immune response.

The term “CRAC channel”, as used herein, denominates the calcium release-activated calcium channel, which belongs to the group of store-operated channels.

The term “puncta”, as used herein, describes specific areas where the STIM1 and/or Orai1 accumulate after depletion of intracellular calcium stores. This occurs at ER-plasma membrane junctions where the endoplasmic reticulum comes in close proximity to the plasma membrane.

The term “heterologous nucleic acid”, as used herein, describes a nucleic acid that originates from a source foreign to the particular host cell, or, if from the same source, that is modified from its original form.

A “modulator” is an agent that modulates an activity of a given polypeptide or protein, e.g., an Orai and/or Stim polypeptide or protein. Modulators of interest include small organic molecules, and are generally within the structures set forth in Formulas I-XIII described herein.

The term “modulate” with respect to such a polypeptide or protein refers to a change in an activity or property of the polypeptide or protein. For example, modulation can cause an increase or a decrease in polypeptide or protein activity, binding characteristic (e.g., binding between Orai and Stim), or any other biological, functional, or immunological property of such a polypeptide or protein. The change in activity can arise from a change in activity of the polypeptide or protein itself, from steric hindrance, binding site inactivation, etc. The terms “protein(s)” and “polypeptide(s)” are used interchangeably.

The terms “contact”, “contacts”, “contacting” have their normal meaning and refer to combining two or more entities (e.g., two proteins, a polynucleotide and a cell, a cell and a candidate agent, etc.). Contacting can occur in vitro, in situ or in vivo and is used interchangeably with “expose to”, “exposed to”, “exposing to.”

As used herein, the terms “reduce”, “decrease” and “inhibit” are used together because it is recognized that, in some cases, an observed activity can be reduced below the level of detection of a particular assay. As such, it may not always be clear whether the activity is “reduced” or “decreased” below a level of detection of an assay, or is completely “inhibited”. As used herein, compounds which are “commercially available” may be obtained from standard commercial sources including Acros Organics (Geel Belgium), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.); Molecular Probes (Eugene, Oreg.); Invitrogen (Carlsbad, Calif.), Applied Biosystems, Inc. (Foster City, Calif.), Glen Research (Sterling, Va.), Biosearch Technologies (Novato, Calif.), Anaspec (Fremont, Calif.) and Berry & Associates (Dexter, Mich.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. The term lower alkyl will be used herein as known in the art to refer to an alkyl, straight, branched or cyclic, of from about 1 to 6 carbons.

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Acyl” refers to a —C(O)R group, where R is hydrogen, alkyl, alkenyl, cycloalkyl, heterocycloalkyl, aryl, arylalkyl, heteroalkyl, heteroalkenyl, or heteroaryl as defined herein. Representative examples include, but are not limited to, formyl, acetyl, cyclohexylcarbonyl, cyclohexylmethylcarbonyl, benzoyl, benzylcarbonyl and the like.

“Acylamino” refers to a —NR′C(O)R group, where R′ is hydrogen, alkyl, cycloalkyl, heterocycloalkyl, aryl, arylalkyl, heteroalkyl, heteroaryl, heteroarylalkyl and R is hydrogen, alkyl, alkoxy, cycloalkyl, heterocycloalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)H, —OC(O)-alkyl, —OC(O)-aryl or —OC(O)-cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkynyl and alkynylene. Lower aliphatic groups typically have from 1 or 2 to 6 or 12 carbon atoms.

“Alkenyl” refers to monovalent olefinically unsaturated hydrocarbyl groups having up to about 11 carbon atoms, such as from 2 to 8 carbon atoms, and including from 2 to 6 carbon atoms, which can be straight-chained or branched and having at least 1 and including from 1 to 2 sites of olefinic unsaturation. Particular alkenyl groups include ethenyl (—CH═CH2), n-propenyl (—CH2CH═CH2), isopropenyl (—C(CH3)=CH2), vinyl and substituted vinyl, and the like.

“Alkoxy” refers to the group —O-alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Alkoxycarbonylamino” refers to the group —NRC(O)OR′ where R is hydrogen, alkyl, aryl or cycloalkyl, and R′ is alkyl or cycloalkyl.

“Alkyl” refers to monovalent saturated aliphatic hydrocarbyl groups particularly having up to about 12 or 18 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Alkylene” refers to divalent saturated aliphatic hydrocarbyl groups particularly having up to about 12 or 18 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkynyl” refers to acetylenically unsaturated hydrocarbyl groups particularly having up to about 12 or 18 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Amino” refers to the radical —NH₂.

“Aminocarbonyl” refers to the group —C(O)NRR where each R is independently hydrogen, alkyl, aryl or cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalky, or where the R groups are joined to form an alkylene group.

“Aralkyl” or “arylalkyl” refers to an alkyl group, as defined above, substituted with one or more aryl groups, as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some cases, an aryl group includes from 6 to 14 carbon atoms.

“Aryloxy” refers to —O-aryl groups wherein “aryl” is as defined herein.

“Azido” refers to a —N₃ group.

“Carbonyl” refers to —C(O)— groups, for example, a carboxy, an amido, an ester, a ketone, or an acyl substituent.

“Carboxyl” refers to a —C(O)OH group

“Cyano” refers to a —CN group.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl.

“Halogen” or “halo” refers to fluoro, chloro, bromo and iodo.

“Hetero” when used to describe a compound or a group present on a compound means that one or more carbon atoms in the compound or group have been replaced by, for example, a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g., heterocycloalkenyl, cycloheteroalkenyl, e.g., heterocycloheteroalkenyl and the like having from 1 to 5, and particularly from 1 to 3 heteroatoms. A heteroatom is any atom other than carbon or hydrogen and is typically, but not exclusively, nitrogen, oxygen, sulfur, phosphorus, boron, chlorine, bromine, or iodine.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. The heteroaryl group can be a 5-20 membered heteroaryl, or 5-10 membered heteroaryl. Particlar heteroaryl groups are those derived from thiophen, pyrrole, benzothiophene, benzofuran, indole, pyridine, quinoline, imidazole, oxazole and pyrazine.

“Heterocycle” refers to organic compounds that contain a ring structure containing atoms in addition to carbon, such as sulfur, oxygen or nitrogen, as part of the ring. They may be either simple aromatic rings or non-aromatic rings. Examples include azoles, morpholine, piperazine, pyridine, pyrimidine and dioxane. The maximum number of heteroatoms in a stable, chemically feasible heterocyclic ring, whether it is aromatic or non aromatic, is determined by factors such as, the size of the ring, the degree of unsaturation and the valence of the heteroatoms. In general, a heterocyclic ring may have one to four heteroatoms so long as the heteroaromatic ring is chemically feasible and stable.

“Hydroxyl” refers to a —OH group.

“Stereoisomer” as it relates to a given compound refers to another compound having the same molecular formula, wherein the atoms making up the other compound differ in the way they are oriented in space, but wherein the atoms in the other compound are like the atoms in the given compound with respect to which atoms are joined to which other atoms (e.g. an enantiomer, a diastereomer, or a geometric isomer). See for example, Morrison and Boyd, Organic Chemistry, 1983, 4th ed., Allyn and Bacon, Inc., Boston, Mass., p. 123.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂. Substituents of interest may include, but are not limited to, —X, —R⁸ (with the proviso that R⁸ is not hydrogen), —O—, ═O, —OR⁸, —SR⁸, —S—, ═S, —NR⁸R⁹, ═NR⁸, —CX₃, —CF₃, —CN, —OCN, —SCN, —NO, —NO₂, ═N₂, —N₃, —S(O)₂O—, —S(O)₂OH, —S(O)₂R⁸, —OS(O₂)O—, —OS(O)₂R⁸, —P(O)(O—)₂, —P(O)(OR⁸)(O—), —OP(O)(OR⁸)(0R⁹), —C(O)R⁸, —C(S)R⁸, —C(O)OR⁸, —C(O)NR⁸R⁹, —C(O)O—, —C(S)OR⁸, —NR¹⁰C(O)NR⁸R⁹, vNR¹⁰C(S)NR⁸R⁹, —NR¹¹C(NR¹⁰)NR⁸R⁹ and —C(NR¹⁰)NR⁸R⁹, where each X is independently a halogen.

“Sulfonyl” refers to the group —SO₂—. Sulfonyl includes, for example, methyl-SO₂—, phenyl-SO₂—, and alkylamino-SO₂—.

“Sulfinyl” refers to the group —S(O)—.

“Thioalkoxy” refers to the group —S-alkyl.

“Thioaryloxy” refers to the group —S-aryl.

“Thioketo” refers to the group ═S.

“Thiol” refers to the group —SH.

“Thio” refers to the group —S—. Thio includes, for example, thioalkoxy, thioaryloxy, thioketo and thiol.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A subject compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

The term “pharmaceutically acceptable salt” means a salt which is acceptable for administration to a patient, such as a mammal (e.g., salts having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

The term “salt thereof” means a compound formed when the hydrogen of an acid is replaced by a cation, such as a metal cation or an organic cation and the like. Where applicable, the salt is a pharmaceutically acceptable salt, although this is not required for salts of intermediate compounds that are not intended for administration to a patient.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereoisomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

### DETAILED DESCRIPTION OF THE EMBODIMENTS

As summarized above, aspects of the invention include store operated calcium channel modulating compounds. The store operated calcium channel modulating compounds are compounds that modulate calcium influx in a cell upon contact with a cell or components thereof. In some instances, the types of cells in which the compounds of the invention exhibit activity are ones that include a CRAC channel.

Methods of the invention include contacting a cell with an effective dose of store operated calcium channel modulator described herein, to inhibit the activity of the store operated calcium channel. In certain embodiments, the store operated calcium channel means that the activity of the channel is inhibited or reduced. In certain embodiments, the expression of transcription factors which are important for T cell activation in reduced, or even prevented. Methods of the invention also include methods of reducing T cell activation in vitro or in vivo, the methods comprising contacting a population of cells comprising T cells with an effective dose of a store operated calcium channel inhibitor. In vitro cultures of T cells include, for example, models of inflammatory, cytotoxic, or atopic responses, for example for pre-clinical testing purposes, compound screening, and the like. In vivo T cells include animal models and human patients for which a reduction in activation of T cells is desired, e.g. to reduce or prevent inflammatory responses, to reduce or prevent graft rejection, to reduce or prevent atopic responses; and the like.

In certain embodiments, the compounds of the invention modulate the calcium influx in a cell by binding to a member of the STIM and Orai family of proteins. Of particular interest are compounds that bind to at least one minimal functional domain of STIM or Orai1, or a complex thereof, by a SMM binding assay such that compounds of interest have a composite Z score of 0 or greater, such as 1 or greater, 1.5 or greater, 2 or greater, 2.5 or greater, 3 or greater, or even greater.

In certain embodiments, the compounds of the invention modulate calcium influx into a cell, which may be measured, for example, by an NFAT coupled luciferase assay that measures functional inhibition of SOC. For example, compounds of the invention inhibit a promoter dependent production of luciferase that is directly activated by SOC in T lymphocytes, relative to a control, i.e. compounds of the invention show signals that are reduced by at least 10%, such as at least 20%, at least 30%, at least 40%, at least 50%, at least 60%, at least 70%, at least 80%, at least 90%, at least 95%, or even more, relative to a control signal. In particular embodiments, compounds of the invention inhibit calcium influx into a cell by an NFAT coupled luciferase assay with an EC50 value of 10 μM or less, such as 3 μM or less, 1 μM or less, 300 nM or less,100 nM or less, or even smaller.

In particular embodiments, the compounds of the invention are inhibitors of STIM-Orai interaction and may also inhibit Ca²⁺ entry through store-operated channels. In particular embodiments, the compounds of the invention are enhancers of STIM-Orai interactions and may also activate Ca²⁺ entry through store-operated channels.

In certain embodiments, the compounds of the invention modulate SOC dependent intracellular calcium levels, and may be assessed by an assay that visualizes calcium using Fura2-AM, a known ratiometric calcium indicator. Compounds of the invention modulate SOC dependent intracellular calcium influx, for example as measured by comparison of influx slope relative to a control.

In certain embodiments, the compounds of the invention modulate expression of transcription factors related to T cell activation, which may be determined, for example, by an assay that measures the mRNA levels of genes in compound treated T lymphocyte cells.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing those components that are described in the publications that might be used in connection with the presently described invention.

### Compositions

As summarized above, aspects of the invention include a store operated calcium channel modulating compound. A store operated calcium channel modulating compound may include a chromenone, an octahydro-naphthalene, a dibenzo-diheteropin-11-one, a 3a, 7a-dihydro-4H-benzofuran-5-one, a tetrahydro-furo[3,4-c]furan-1-one, or a 3,3a,4,5-tetrahydro-2H-benzofuran-6-one, for example as set forth in the following structures I-XIII.

In certain embodiments, a substituent may contribute to optical isomerism and/or stereo isomerism of a compound. Salts, solvates, hydrates, and prodrug forms of a compound are also of interest. All such forms are embraced by the present invention. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

The following are examples of compounds of the invention.

### Chromenones

In certain embodiments, the store operated calcium channel modulating compounds are chromenones, e.g., an aryl substituted chromen-4-one further substituted with substituents such as hydrogen, an aliphatic, an alkoxy, an acyloxy, an aryloxy, an amino, an aryl, a carbonyl, cyano, hydroxyl, a halogen, a heterocycle, a thiol or nitro. The aryl substituent bond may be to the 3 position of the chromen-4-one scaffold. In certain embodiments, the chromenone is a 3-phenyl-chromen-4-one compound. Substituent bonds to the chromenone may be to the carbons of the rings, e.g., substituent bonds may be to the 3-, 5-, and 7-positions; the 2-, 3-, 5-, and 7-positions; the 3-, 6-, 7-, and 8-positions; or the 3-, 7-, and 8-positions of the chromen-4-one scaffold.

In certain embodiments, a chromenone compound of the invention is of the structure of formula (I):

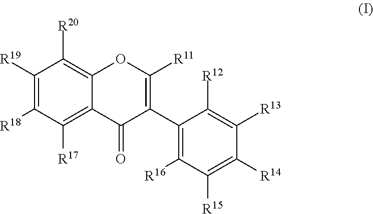

_{where R}11, R¹², R¹³, R¹⁴, R¹⁵, R¹⁶, R¹⁷, R¹⁸, R¹⁹ and R²⁰ are independently selected from hydrogen, an aliphatic, an alkoxy, an acyloxy, an aryloxy, an amino, an aryl, a carbonyl, cyano, hydroxyl, a halogen, a heterocycle, a thiol and nitro, where optionally two of R¹², R¹³, R¹⁴, R¹⁵, R¹⁶, R¹⁷, R¹⁸, R¹⁹ and R²⁰ can be cyclically linked;

In particular embodiments, in formula (I), at least one of R¹⁷, R¹⁸, R¹⁹ and R²⁰ is hydrogen; and at least two of R¹², R¹³, R¹⁴, R¹⁵ and R¹⁶ is hydrogen.

In particular embodiments, in formula (I), R¹², R¹³, R¹⁴, R¹⁵, R¹⁶, R¹⁷, R¹⁸, R¹⁹ and R²⁰ is are independently selected from hydrogen, an alkyl, an alkoxy, an acyloxy, an aryloxy and hydroxyl. In particular embodiments, in formula (I), R¹¹ is selected from hydrogen and a carbonyl (e.g., an alkoxycarbonyl).

In certain embodiments, a chromenone compound of the invention is of the structure of formula (II):

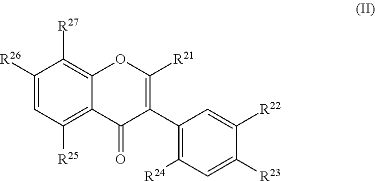

where R²¹, R²², R²³, R²⁴, R²⁵, R²⁶ and R²⁷ are independently selected from hydrogen, an aliphatic, an alkoxy, an acyloxy, an aryloxy, an amino, an aryl, a carbonyl, cyano, hydroxyl, a halogen, a heterocycle, a thiol and nitro, where optionally two of R²², R²³, R²⁴, R²⁵, R²⁶ and R²⁷ can be cyclically linked.

In particular embodiments, in formula (II), R²², R²³, R²⁴, R²⁵, R²⁶ and R²⁷ are independently selected from hydrogen, an alkyl, an alkoxy, an acyloxy, an aryloxy and hydroxyl, where optionally R²² and R²³ and/or R²⁶ and R²⁷ can be cyclically linked. In particular embodiments, in formula (II), R²¹ is selected from hydrogen and a carbonyl (e.g., an alkoxycarbonyl).

In particular embodiments, in formula (II), R²³, R²⁵ and R²⁶ are independently selected from hydroxyl, an acyloxy, an aryloxy and an alkoxy.

In particular embodiments, in formula (II), and R²², R²⁴ and R²⁷ are hydrogen.

In particular embodiments, a compound of the invention is of one of the following structures:

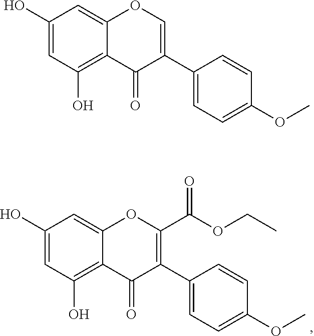 [SMILES: COc1ccc(-c2coc3cc(O)cc(O)c3c2=O)cc1 ; CCOC(=O)c1oc2cc(O)cc(O)c2c(=O)c1-c1ccc(OC)cc1]

or a substituted version thereof.

In certain embodiments, a chromenone compound of the invention is of the structure of formula (III):

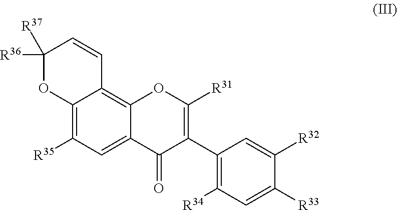

where R³¹, R³², R³³, R³⁴ and R³⁵ are independently selected from hydrogen, an aliphatic, an alkoxy, an acyloxy, an aryloxy, an amino, an aryl, a carbonyl, cyano, hydroxyl, a halogen, a heterocycle, a thiol and nitro; and

R³⁶ and R³⁷ are independently selected from hydrogen, an aliphatic, an alkoxy, an acyloxy, an aryloxy, an amino, an aryl, a carbonyl, cyano, hydroxyl, a heterocycle and a thiol.

In particular embodiments, in formula (III), R³², R³³, R³⁴, R³⁵, R³⁶ and R³⁷ are independently selected from hydrogen, an alkyl, an alkoxy, an acyloxy, an aryloxy and hydroxyl, where optionally R³² and R³³ and/or R³⁶ and R³⁷ can be cyclically linked. In particular embodiments, in formula (III), R³¹ is selected from hydrogen and a carbonyl (e.g., an alkoxycarbonyl).

In particular embodiments, in formula (III), R³², R³³, R³⁴ and R³⁵ are independently selected from hydroxyl, an acyloxy, an aryloxy and an alkoxy, where optionally R³² and R³³ can be cyclically linked.

In particular embodiments, in formula (III), R³⁶ and R³⁷ are independently selected from hydrogen and a lower alkyl.

In particular embodiments, a compound of the invention is of one of the following structures:

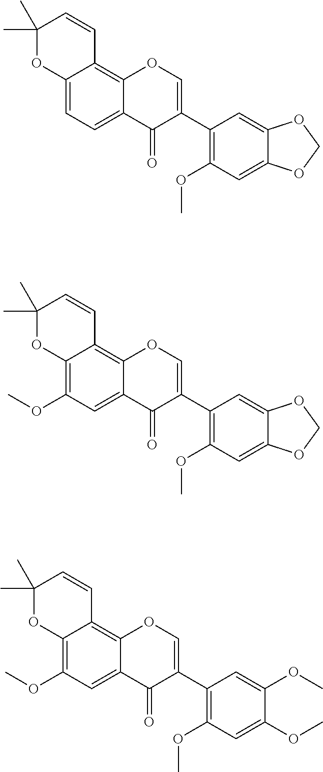

or a substituted version thereof.

### Octahydro-Naphthalenes

In certain embodiments, the store operated calcium channel modulating compounds are octahydro-naphthalenes, e.g., a substituted 1,2,3,4,5,6,7,8-octahydro-naphthalene-1,2-diol compound.

In certain embodiments, an octahydro-naphthalene compound of the invention is of the structure of formula (IV):

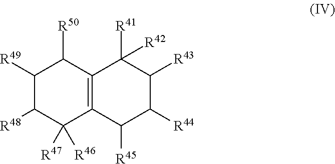

where R⁴¹, R⁴², R⁴³, R⁴⁴, R⁴⁵, R⁴⁶, R⁴⁷, R⁴⁸, R⁴⁹ and R⁵⁰ are independently selected from hydrogen, an aliphatic, an alkoxy, an acyloxy, an aryloxy, an amino, an aryl, a carbonyl, cyano, hydroxyl, a heterocycle and a thiol .

In particular embodiments, in formula (IV), R⁴¹, R⁴², R⁴³, R⁴⁴, R⁴⁵, R⁴⁶, R⁴⁷, R⁴⁸, R⁴⁹ and R⁵⁰ are independently selected from hydrogen, an aliphatic, an alkyl (e.g., a lower alkyl), an alkoxy, an acyloxy (e.g., an acetyl), an aryloxy, and hydroxyl. In particular embodiments, in formula (IV), R⁴¹ is a hydroxy and alkenyl substituted alkyl group; and R⁴², R⁴³, R⁴⁴, R⁴⁵, R⁴⁶, R⁴⁷, R⁴⁸, R⁴⁹ and R⁵⁰ are independently selected from hydrogen, an alkyl (e.g., a lower alkyl), an alkoxy, an acyloxy (e.g., an acetyl) and hydroxyl.

In certain embodiments, a compound of the invention is of the structure of formula (V):

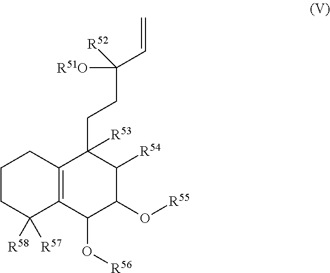

where R⁵¹, R⁵², R⁵³, R⁵⁴, R⁵⁵, R⁵⁶, R⁵⁷ and R⁵⁸ are independently selected from hydrogen, an aliphatic, an alkoxy, an acyloxy, an aryloxy, an amino, an aryl, a carbonyl, cyano, hydroxyl, a heterocycle and a thiol.

In particular embodiments, in formula (V), R⁵¹, R⁵⁵ and R⁵⁶ are independently selected from hydrogen, an alkyl and an acyl.

In particular embodiments, in formula (V), R⁵², R⁵³, R⁵⁴, R⁵⁷ and R⁵⁸ are independently selected from hydrogen and an alkyl.

In particular embodiments, a compound of the invention is of the following structure:

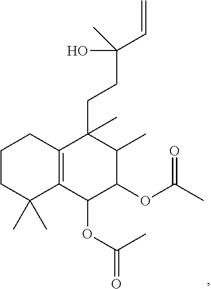 [SMILES: C=CC(C)(O)CCC1(C)C2=C(C(OC(C)=O)C(OC(C)=O)C1C)C(C)(C)CCC2]

or a substituted version thereof.

Dibenzo-diheteropin-11-ones

In certain embodiments, store operated calcium channel modulating compounds of the invention are dibenzo-diheteropin-11-ones, e.g., compounds where the 7membered ring includes a keto group at the 11 position and two heteroatoms at the 5- and 10-positions, where the heteroatoms are independently nitrogen, sulfur or oxygen. In certain embodiments, the dibenzo-diheteropin-11-one compound is a 5H-10-oxa-5-aza-dibenzo[a,d]cyclohepten-11-one, a dibenzo[b,e][1,4]dioxepin-11-one, a 10H-dibenzo[b,f][1,4]oxazepin-11-one or a 5,10-dihydro-dibenzo[b,e][1,4]diazepin-11-one.

In certain embodiments, a dibenzo-diheteropin-11-one compound of the invention is of the structure of formula (VI):

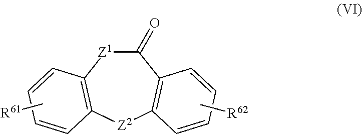

where R⁶¹ and R⁶² are independently one or more groups, each R⁶¹ and R⁶² independently selected from hydrogen, an aliphatic, an alkoxy, an acyloxy, an aryloxy, an amino, an aryl, a carbonyl, cyano, hydroxyl, a halogen, a heterocycle, a thiol and nitro;

Z¹ and Z² are independently selected from O, S and NR, where R is hydrogen or alkyl.

In particular embodiments, in formula (VI), Z¹ and Z² are O, and R⁶¹ and R⁶² are independently one or more groups, each R⁶¹ and R⁶² independently selected from hydrogen, an alkyl, an aryl, a hydroxyl, an alkoxy, an acyloxy, and an aryloxy.

In certain embodiments, a dibenzo-diheteropin-11-one compound of the invention is of the structure of formula (VII):

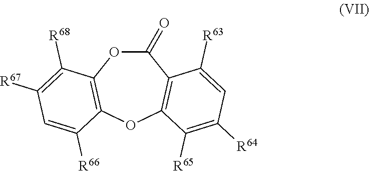

where R⁶³, R⁶⁴, R⁶⁵, R⁶⁶, R⁶⁷ and R⁶⁸ are independently selected from hydrogen, an aliphatic, an alkoxy, an acyloxy, an aryloxy, an amino, an aryl, a carbonyl, cyano, hydroxyl, a halogen, a heterocycle, a thiol and nitro.

In particular embodiments, in formula (VII), at least two of R⁶³, R⁶⁴ and R⁶⁵ are independently a lower alkyl. In particular embodiments, in formula (VII), at least two of R⁶⁶, R⁶⁷ and R⁶⁸ are independently a lower alkyl. In particular embodiments, in formula (VII), at least one of R⁶³, R⁶⁴ and R⁶⁵ is hydroxyl, an alkoxy or an acyloxy. In particular embodiments, in formula (VII), at least one of R⁶⁶, R⁶⁷ and R⁶⁸ is hydroxyl, an alkoxy or an acyloxy.

In particular embodiments, in formula (VII), R⁶³, R⁶⁵, R⁶⁶ and R⁶⁸ are independently selected from hydrogen and a lower alkyl.

In particular embodiments, in formula (VII), R⁶⁴ and R⁶⁷ are independently selected from hydrogen, hydroxyl, an alkoxy and an acyloxy.

In particular embodiments, a compound of the invention is of the following structure:

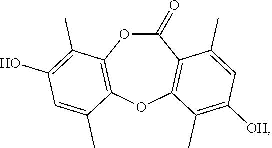 [SMILES: Cc1cc(O)c(C)c2c1Oc1c(C)c(O)cc(C)c1C(=O)O2]

or a substituted version thereof.

### Dihydro-benzofuran-5-one

In certain embodiments, store operated calcium channel modulating compounds of the invention are dihydro-benzofuran-5-ones. In certain embodiments, a dihydro-benzofuran-5-one compound is a substituted 3a,7a-dihydro-4H-benzofuran-5-one.

In certain embodiments, a dihydro-benzofuran-5-one compound is of the structure of formula (VIII):

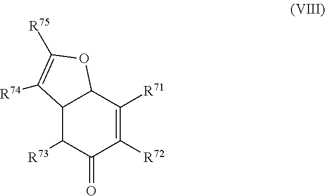

where R⁷¹, R⁷², R⁷³, R⁷⁴ and R⁷⁵ are independently selected from hydrogen, an aliphatic, an alkoxy, an acyloxy, an aryloxy, an amino, an aryl, a carbonyl, cyano, hydroxyl, a halogen, a heterocycle, a thiol and nitro, where optionally two adjacent groups of R⁷¹, R⁷², R⁷³, R⁷⁴ and R⁷⁵ can be cyclically linked.

In particular embodiments, in formula (VIII), R⁷³ and R⁷⁴ can be cyclically linked.

In particular embodiments, in formula (VIII), R⁷⁴ is a carbonyl (e.g., an alkoxycarbonyl). In particular embodiments, in formula (VIII), R⁷³ is a alkylmethylene group (e.g., R—CH═). In particular embodiments, in formula (VIII), R⁷¹ and R⁷² are hydrogen.

In certain embodiments, a benzofuran-5-one is of the structure of formula (IX):

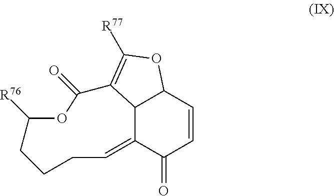

where R⁷⁶ and R⁷⁷ are independently selected from hydrogen, an aliphatic, an alkoxy, an acyloxy, an aryloxy, an amino, an aryl, a carbonyl, cyano, hydroxyl, a heterocycle and a thiol.

In particular embodiments, in formula (IX), R⁷⁶ is an alkyl.

In particular embodiments, in formula (IX), R⁷⁷ is an aliphatic (e.g., a conjugated alkenyl group, optionally substituted with a carboxy group).

In particular embodiments, a compound of the invention is of the following structure:

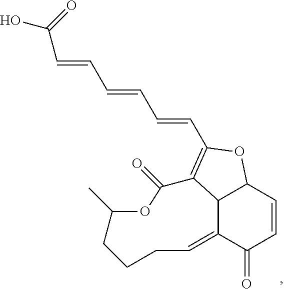 [SMILES: CC1CCC/C=C2/C(=O)C=CC3OC(/C=C/C=C/C=C/C(=O)O)=C(C(=O)O1)C23]

or a substituted version thereof.

Tetrahydrofuro-furan-1-ones

In certain embodiments, store operated calcium channel modulating compounds of the invention are tetrahydrofuro-furan-1-ones. In certain embodiments, a tetrahydrofuro-furan-1-one compound is a substituted tetrahydro-furo[3,4-c]furan-1-one.

In certain embodiments, a tetrahydrofuro-furan-1-one compound is of the structure of formula (X):

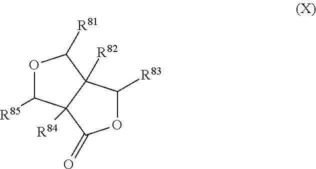

where R⁸¹, R⁸², R⁸³, R⁸⁴ and R⁸⁵ are independently selected from hydrogen, an aliphatic, an alkoxy, an acyloxy, an aryloxy, an amino, an aryl, a carbonyl, cyano, hydroxyl, a heterocycle and a thiol.

In particular embodiments, in formula (X), R⁸³ and R⁸⁵ are independently an aryl (e.g. a phenyl) or a heterocycle. In particular embodiments, in formula (X), R⁸¹, R⁸² and R⁸³ are hydrogen.

In certain embodiments, a compound of the invention is of the structure of formula (XI):

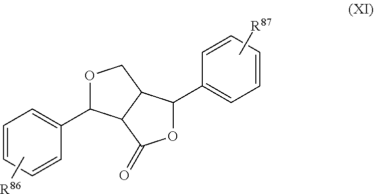

where R⁸⁶ and R⁸⁷ are independently one or groups, each R⁸⁶ and R⁸⁷ independently selected from hydrogen, an aliphatic, an alkoxy, an acyloxy, an aryloxy, an amino, an aryl, a carbonyl, cyano, hydroxyl, a halogen, a heterocycle, a nitro and a thiol.

In particular embodiments, in formula (XI), each R⁸⁶ and R⁸⁷ are independently selected from an alkyl, a hydroxy, an alkoxy and an aryloxy, where two R⁸⁶ groups and/or two R⁸⁷ groups can be cyclically linked.

In particular embodiments, a compound of the invention is of the following structure:

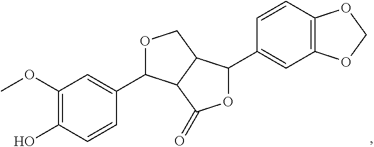 [SMILES: COc1cc(C2OCC3C(c4ccc5c(c4)OCO5)OC(=O)C23)ccc1O]

or a substituted version thereof.

Tetrahydro-benzofuran-6-ones

In certain embodiments, store operated calcium channel modulating compounds of the invention are tetrahydro-benzofuran-6-ones. In certain embodiments, a tetrahydro-benzofuran-6-one compound is a substituted 3,3a,4,5-tetrahydro-2H-benzofuran-6-one.

In certain embodiments, a tetrahydro-benzofuran-6-one compound is of the structure of formula (XII):

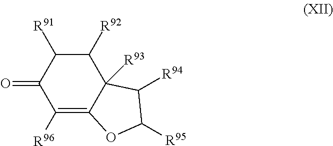

where R⁹¹, R⁹², R⁹³, R⁹⁴, R⁹⁵ and R⁹⁶ are independently selected from hydrogen, an aliphatic, an alkoxy, an acyloxy, an aryloxy, an amino, an aryl, a carbonyl, cyano, hydroxyl, a heterocycle and a thiol.

In particular embodiments, in formula (XII), R⁹¹, R⁹², R⁹³, R⁹⁴, R⁹⁵ and R⁹⁶ are independently selected from hydrogen, an alkyl, an alkoxy, an aryl, a heterocycle, an allyl.

In particular embodiments, in formula (XII), R⁹⁵ is an aryl or a heterocycle. In particular embodiments, in formula (XII), R⁹³ is an allyl. In particular embodiments, in formula (XII), R⁹¹ and R⁹⁶ are independently selected from hydrogen, a hydroxyl, an alkoxy, an aryloxy and an acyloxy.

In certain embodiments, a compound of the invention is of the structure of formula (XIII):

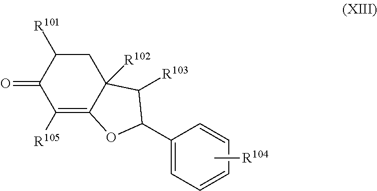

where R¹⁰¹, R¹⁰², R¹⁰³ and R¹⁰⁵ are independently selected from hydrogen, an aliphatic, an alkoxy, an acyloxy, an aryloxy, an amino, an aryl, a carbonyl, cyano, hydroxyl, a heterocycle and a thiol;

R¹⁰⁴ is one or groups, each R¹⁰⁴ independently selected from hydrogen, an aliphatic, an alkoxy, an acyloxy, an aryloxy, an amino, an aryl, a carbonyl, cyano, a halogen, hydroxyl, a heterocycle, nitro and a thiol, where optionally two R¹⁰⁴ groups can be cyclically linked.

In particular embodiments, in formula (XIII), R¹⁰¹, R¹⁰², R¹⁰³, R¹⁰⁵ and each R¹⁰⁴ are independently selected from hydrogen, an alkyl, an allyl, an alkoxy, an acyloxy, an aryloxy and hydroxyl, where optionally any two of R¹⁰¹, R¹⁰², R¹⁰³, R¹⁰⁵ and R¹⁰⁴ can be cyclically linked.

In particular embodiments, in formula (XIII), two adjacent R¹⁰⁴ groups are cyclically linked.

In particular embodiments, a compound of the invention is of the following structure:

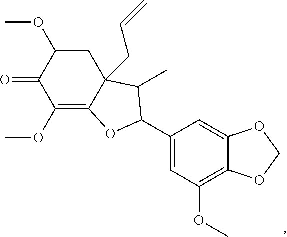 [SMILES: C=CCC12CC(OC)C(=O)C(OC)=C1OC(c1cc(OC)c3c(c1)OCO3)C2C]

or a substituted version thereof.

### Pharmaceutical Preparations

Also provided are pharmaceutical preparations. Pharmaceutical preparations are compositions that include a store operated calcium channel (SOC) modulating compound (for example one or more SOC modulating compounds, either alone or in the presence of one or more additional active agents) present in a pharmaceutically acceptable vehicle, where the preparation may be provided in a unit dose that contains an effective dose of the compound.

The term “pharmaceutically acceptable” means approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention.

The SOC modulating compound can be injected into the host organism. The agent may be directly introduced into the cell (i.e., intracellularly); or introduced extracellularly into a cavity, interstitial space, into the circulation of an organism, introduced orally, etc.

Depending on the nature of the compound, SOC modulating compounds may be administered to the host using any convenient means capable of resulting in the desired modulation of CRAC in the target cell. Thus, the SOC modulating compounds can be incorporated into a variety of formulations for therapeutic administration. More particularly, the SOC modulating compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the SOC modulating compounds can be achieved in various ways, including oral, buccal, rectal, parenteral, intraperitoneal, intradermal, transdermal, intracheal, etc., administration.

Administration of SOC modulating compounds of the invention may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In particular embodiments, the compounds and compositions of the invention are administered orally. In particular embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The SOC modulating compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (i.e., sunblocking agents), etc.

For use in wound healing or treatment of other acute or chronic conditions of the epidermis, a compound of the invention may be formulated for topical administration. The vehicle for topical application may be in one of various forms, e.g. a lotion, cream, gel, ointment, stick, spray, or paste. They may contain various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant to disperse and suspend the discontinuous phase within the continuous phase.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Preferred dosages for a given compound are readily determinable by those of skill in the art by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the SOC modulating compounds may be administered in the form of a free base, their pharmaceutically acceptable salts, or they may also be used alone or in appropriate association, as well as in combination, with other pharmaceutically active compounds.

### Methods of Use

Aspects of the invention include methods of using SOC modulating compounds, e.g., as described above, to modulating the activity of SOC channels in a cell. In practicing methods of the invention, the cells of interest are contacted with an effective amount of a SOC modulating compound, e.g., as described above. By effective amount is meant an amount of the SOC modulating compound that is sufficient to modulate the activity of a SOC in the target cell population to a desired level.

In practicing methods of the invention, the cells of interest may be contacted with the effective amount of the SOC modulating compound in an in vitro or ex vivo culture system, or in vivo. For example, a SOC modulating compound may be contacted to primary cells grown under standard tissue culture conditions or alternatively to cells that are part of a whole animal (e.g., administered to a subject). As such, the target cell or collection of cells may vary, where the collection of cells may be cultured cells, a whole animal or portion thereof, e.g., tissue, organ, etc. As such, the target cell(s) may be a host animal or portion thereof.

In the subject methods, the SOC modulating compound may be contacted with the target cells using any convenient protocol that results in the desired level of SOC channel activity. Thus, the SOC modulating compound can be incorporated into a variety of pharmaceutical compositions for therapeutic administration, e.g., as described above. For example, the SOC modulating compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols, such as described above. As such, administration of the SOC modulating compound can be achieved in various ways, including oral, buccal, rectal, parenteral, intraperitoneal, intradermal, transdermal, intracheal, etc., administration.

The subject methods find use in the treatment of a variety of different conditions in which modulation of activity of a SOC channel in the host is desired. By treatment is meant that at least an amelioration of the symptoms associated with the condition afflicting the host is achieved, where amelioration is used in a broad sense to refer to at least a reduction in the magnitude of a parameter, e.g. symptom (such as inflammation), associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The SOC modulating compounds, e.g., as described above, find use in a variety of applications. Applications of interest include, but are not limited to: therapeutic applications, research and manufacturing applications, and screening applications. Each of these different applications are now reviewed in greater details below.

### Therapeutic Applications

SOC modulating compounds of the invention find use in a variety of therapeutic applications. Therapeutic applications of interest include those applications in which increased or reduced activity of a SOC is the cause or a compounding factor in disease progression. As such, the subject compounds find use in the treatment of a variety of different conditions in which modulation of the activity of a SOC in the host is desired. Examples of disease conditions/disorders and therapeutic applications which may be treated with compounds of the invention include, but are not limited to: disorders related to the immune system, immunosuppression, inflammation, cancer and other proliferative diseases, preventing cancer metastasis, liver diseases and disorders, and kidney diseases, stroke and thrombosis, preventing or treating autoimmune disease and allergy, and skin disorders.

Examples of disorders related to inflammation which may be treated with compounds of the invention include asthma, chronic obstructive pulmonary disease, rheumatoid arthritis, inflammatory bowel disease, glomerulonephritis, neuroinflammatory diseases such as multiple sclerosis.

Examples of immune disorders which may be treated with compounds of the invention include psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis

One application where SOC modulating compounds of the invention find use is in immunosuppression.

Examples of disorders related to cancer and other proliferative diseases which may be treated with compounds of the invention include malignancies of lymphoreticular origin, bladder cancer, breast cancer, colon cancer, endometrial cancer, head and neck cancer, lung cancer, melanoma, ovarian cancer, prostate cancer and rectal cancer.

Examples of hepatic or liver diseases and disorders include liver injury, for example, due to transplantation, hepatitis and cirrhosis, chronic liver disease and transplantation injury after cold preservation-warm reoxygenation.

Kidney or renal diseases and disorders may be treated with compounds of the invention, e.g., where mesangial cell hyperplasia is often a key feature of such diseases and disorders. Such diseases and disorders may be caused by immunological or other mechanisms of injury, including IgAN, membranoproliferative glomerulonephritis or lupus nephritis. Imbalances in the control of mesangial cell replication also appear to play a key role in the pathogenesis of progressive renal failure. Modulators of store-operated calcium influx may aid in the treatment of glomerular diseases by inhibiting mesangial cell proliferation.

Modulation of T cell Activation

Increasing CRAC channel activation in T cells augments the output of T cell receptor signaling, as indicated, inter alia, by the elevation of intracellular calcium, cytokine production, and cell proliferation. Inhibiting CRAC channel activation inhibits T cell receptor signaling.

Introduction of an effective amount of a SOC modulating compound into a mammalian cell as described above results in a modulation of T cell activation.

The above described methods work in any mammalian cell, where representative mammal cells of interest include, but are not limited to cells of: ungulates or hooved animals, e.g., cattle, goats, pigs, sheep, etc.; rodents, e.g., hamsters, mice, rats, etc.; lagomorphs, e.g., rabbits; primates, e.g., monkeys, baboons, humans, etc.; and the like.

Before, during, or after treatment, the host may be assessed for immune responsiveness to a candidate antigen by various methods known in the art. The diagnosis may determine the level of reactivity, e.g. based on the number of reactive T cells found in a sample, as compared to a negative control from a naive host, or standardized to a data curve obtained from one or more patients. In addition to detecting the qualitative and quantitative presence of reactive T cells, the T cells may be typed as to the expression of cytokines known to increase or suppress inflammatory responses. It may also be desirable to type the epitopic specificity of the reactive T cells.

T cells may be isolated from patient peripheral blood, lymph nodes, or preferably from the site inflammation. Reactivity assays may be performed on primary T cells, or the cells may be fused to generate hybridomas. Such reactive T cells may also be used for further analysis of disease progression, by monitoring their in situ location, T cell receptor utilization, etc. Assays for monitoring T cell responsiveness are known in the art, and include proliferation assays and cytokine release assays.

Proliferation assays measure the level of T cell proliferation in response to a specific antigen, and are widely used in the art. In an exemplary assay, patient lymph node, blood or spleen cells are obtained. A suspension of from about 104 to 107 cells, usually from about 105 to 106 cells is prepared and washed, then cultured in the presence of a control antigen, and test antigens. The test antigens may be any peptides of interest. The cells are usually cultured for several days. Antigen-induced proliferation is assessed by the monitoring the synthesis of DNA by the cultures, e.g. incorporation of 3H-thymidine during the last 18 H of culture.

Enzyme linked immunosorbent assay (ELISA) assays are used to determine the cytokine profile of reactive T cells, and may be used to monitor for the expression of such cytokines as IL-2, IL-4, IL-5, γIFN, etc. The capture antibodies may be any antibody specific for a cytokine of interest, where supernatants from the T cell proliferation assays, as described above, are conveniently used as a source of antigen. After blocking and washing, labeled detector antibodies are added, and the concentrations of protein present determined as a function of the label that is bound.

Where the agent is acting to decrease T cell activation, the net effect is to increase the threshold for antigen signaling, and to decrease the sensitivity of a T cell to antigen, and conversely for an increase in T cell activation. The effect may be mediated in mature T cells, e.g. non-naïve T cells that have been exposed to an antigen of interest. Alternatively the target cell may be a progenitor to such mature T cells. Conditions of interest for downregulating T cells responses include allergic responses, autoimmune diseases, and in conjunction with transplantation, where graft rejection may occur as a result of T cell mediated immune responses.

Immune related diseases include: autoimmune diseases in which the immune response aberrantly attacks self-antigens, examples of which include but are not limited to multiple sclerosis (MS), acute disseminated encephalomyelitis (ADEM), rheumatoid arthritis (RA), type I autoimmune diabetes (IDDM), atherosclerosis, systemic lupus erythematosus (SLE), anti-phospholipid antibody syndrome, Guillain-Barre syndrome (GBS) and its subtypes acute inflammatory demyelinating polyradiculoneuropathy, and the autoimmune peripheral neuropathies; allergic diseases in which the immune system aberrantly attacks molecules such as pollen, dust mite antigens, bee venom, peanut oil and other foods, etc.; and tissue transplant rejection in which the immune system aberrantly attacks antigens expressed or contained within a grafted or transplanted tissue, such as blood, bone marrow cells, or solid organs including hearts, lungs, kidneys and livers; and the immune response against tumors. Samples are obtained from patients with clinical symptoms suggestive of an immune-related disease or with an increased likelihood for developing such a disease based on family history or genetic testing.

Other immune related diseases include allergy, or hypersensitivity, of the immune system, including delayed type hypersensitivity and asthma. Most cases of “atopic” or “allergic” asthma occur in subjects whom also exhibit immediate hypersensitivity responses to defined environmental allergens, and challenge of the airways of these subjects with such allergens can produce reversible airway obstruction. Both T cells and mast cells (and other FcRI+ cells) can have effector cell and immunoregulatory roles in these disorders.

NKT cells constitute a lymphocyte subpopulation that are abundant in the thymus, spleen, liver and bone marrow and are also present in the lung. They develop in the thymus from the CD4+CD8+ progenitor cells and circulate in the blood, have distinctive cytoplasmic granules, and can be functionally identified by their ability to kill certain lymphoid tumor cell lines in vitro without the need for prior immunization or activation. The mechanism of NKT cell killing is the same as that used by the cytotoxic T cells generated in an adaptive immune response; cytotoxic granules are released onto the surface of the bound target cell, and the effector proteins they contain penetrate the cell membrane and induce programmed cell death. There is evidence that suggests NKT cells are involved in the pathogenesis of conditions including asthma and certain autoimmune diseases.

Where a patient is undergoing transplantation, it may be desirable to downregulate generally or specifically the patient immune response. In such cases, the therapeutic agent may be introduced prior to, concurrently with, or following the transplantation.

Where the SOC modulating compound is acting to increase expression CRAC activation, the net effect is to increase the sensitivity of a T cell to antigen. Conditions of interest for upregulating T cell responsiveness include conditions where there is an inadequate immune response, e.g. in the induction of immune responsiveness to cancer, to chronically infected cells, and the like.

Increased activation finds use in eliciting an immune response in an autologous, allogeneic or xenogeneic host. For example, where a tumor cell or a chronically infected cell expresses a protein, or over-expresses the protein relative to normal cells, a cytolytic immune response may be induced, where the tumor cell or infected cell is preferentially killed. The antigen for such purposes may be from the same or a different species. As used herein, the term antigen is intended to refer to a molecule capable of eliciting an immune response in a mammalian host, which may be a humoral immune response, i.e. characterized by the production of antigen-specific antibodies, or a cytotoxic immune response, i.e. characterized by the production of antigen specific cytotoxic T lymphocytes. The SOC modulating compound is administered in combination with the tumor antigen.

Several methods exist which can be used to induce an immune response against weakly antigenic protein, i.e. autologous proteins, etc. The immunogen is usually delivered in vivo to elicit a response, but in some cases it is advantageous to prime antigen presenting cells, e.g. dendritic cells, ex vivo prior to introducing them into the host animal.

In one embodiment, polypeptide antigens are mixed with an adjuvant that will augment specific immune responses to the antigen, wherein the adjuvant comprises a SOC modulating compound that increases CRAC activity in the targeted cell. Vaccine antigens may be presented using microspheres, liposomes, may be produced using an immunostimulating complex (ISCOM), as is known in the art.

### Screening Applications

The screening methods, e.g., as described above, find use in a variety of applications, including identifying and/or testing candidate SOC modulating compounds use in a wide range of research and therapeutic applications, such as pharmaceutical development, manufacturing, and quality assurance/control, as well as treating conditions in a subject. Applications of interest include use of the screening methods of the invention for performing research, as well as for pharmaceutical compliance related to GLP (“Good Laboratory Practice”) and GMP (“Good Manufacturing Practice” also referred to as “cGMP” or “current Good Manufacturing Practice”)) and laboratory services. Thus the screening methods of the invention find broad use in research and lead development, sample analysis, as well as assay development, validation, drug regulatory submissions and compliance for new drug substances and drug products, drug product release and compound auditing in general. By “compound auditing” is meant quality assurance and/or quality control of a compound.

Compound auditing in accordance with the subject screening methods may be exploited in multiple settings. One example is in assay development or simply to transfer an assay from one location to another, whether or not it requires GLP and/or GMP compliance. This aspect may include the use of the subject screening methods to ensure that a compound of interest performs consistently and provides continuity in an assay over time. Statistical data analysis and related relevant data analysis tools can be exploited to best match the compound and use of interest. For instance, the screening method can be performed under “research level” protocols to identify those parameters such as the limit of detection (LOD), the limit of quantitation (LOQ) and the linear range necessary for assay validation and/or transfer. As such, the screening methods find use in compiling and executing SOPs (“Standard Operating Procedure” or “Standard Operating Protocol”) which can be used for compound auditing.

Additional uses of the screening methods of the invention include the generation and/or execution one or more GLP or GMP protocols that assess one or more of linearity, accuracy, precision, specificity, robustness, ruggedness and system suitability for one or more compounds of interest for a given end use. Generation of such protocols may include assays for identifying as well as testing of a compound of interest, including QA and/or QC, as well as generating controls that may be aliquoted under GLP or GMP compliance which may be used over several years depending upon the stability of the compound of interest.

The subject screening methods may be used in qualitative and/or quantitative potency assays for routine lot release, lot comparisons, sampling, and stability assessment of a compound of interest.

The screening methods may also be used in a multiple assay approach (i.e., assay matrix), such as when it is desirable to develop or use more than a single assay (e.g., an assay matrix often finds use when there is limited knowledge of product and mechanism of action, the product has multiple components with multiple biological activities, time is constrained due to limited product stability, biological assay is not quantitative and the like). Thus the subject screening methods may find use in a combination of assays where the combined results constitute an acceptable product release and/or potency assay (e.g., a quantitative physical assay along with a qualitative bioassay).

### Systems and Kits

Also provided are systems and kits that include compounds of the invention.

Systems of the invention are collections of active agents brought together, e.g., by a health care practitioner, for administration to a subject, such as a patient. Such systems may include a SOC modulating compound of the invention and one or more additional active agents. Kits that include SOC modulating compounds of the invention are also provided. Kits of the invention may include one or more dosages of a SOC modulating compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to reduce T cell activation.

These instructions may be present in the subject systems and kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

A novel assay was used to identify the intracellular domains of Stim1 and Orai1 that are important for the function of the channels. These domains were used to screen an 80K small molecule library for interacting compounds. Approximately 125 binding compounds were identified and used in functional screens to determine which compounds inhibit calcium influx and prevent the expression of transcription factors that are important for T cell activation. Derivatives of these compounds were identified and characterized for dose response. Additional studies were performed to characterize the mechanism by which they alter store operated calcium influx.

Chemical Binding screen Against Minimal Functional Domains of Stim and Orai

The CAD domain was identified as a domain that activates SOC or CRAC channels by directly binding to the N- and C-termini of Orai. Further, ectopic expression of CAD in cells activates SOC without requiring the depletion of ER stores. Prevention of the binding of the CAD to Orai results in the inability of the channels to activate. CAD along with the N- and C-termini of Orai as the minimal functionally important domains for SOC activation were utilized to identify specific small molecule inhibitors of the channel.

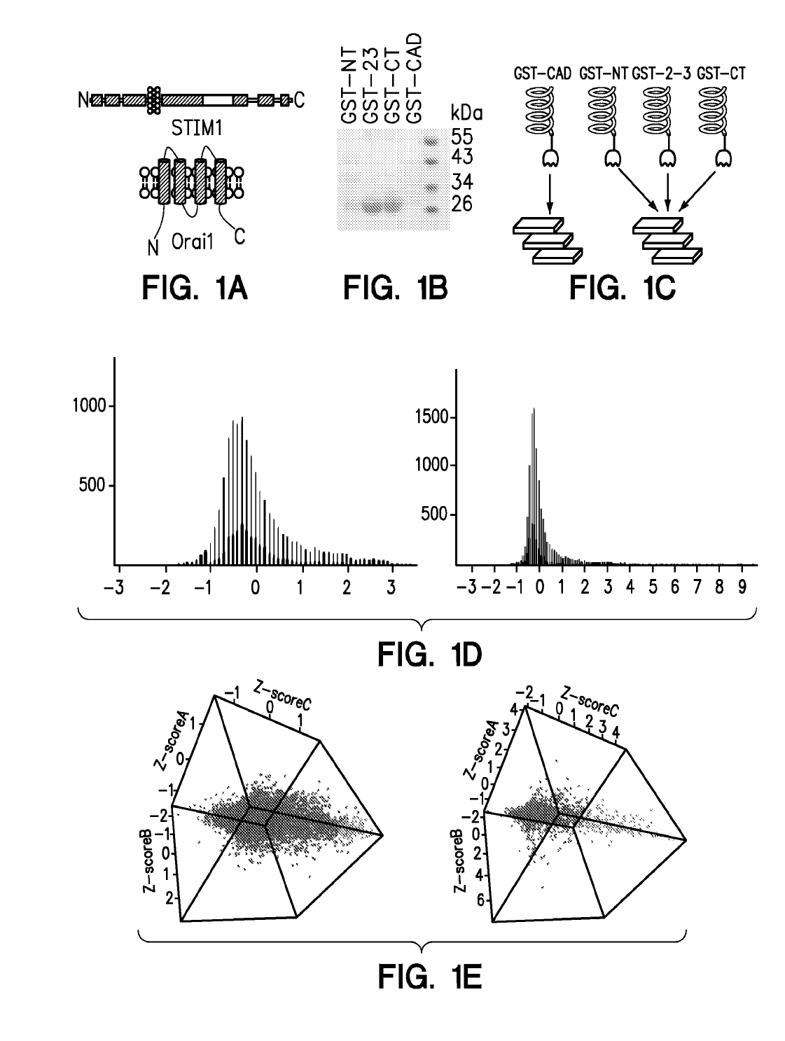

Regions of Stim, CAD, and intracellular domains of Orai were expressed as GST fused domains are represented in red in FIG. 1A, for use in screening for small molecules that bind directly and tightly to these small domains that are functionally required for the activity of the SOC. FIG. 1B shows the SDS PAGE of the GST fused proteins of interest.

A cartoon depiction of the SMM binding screen utilized is shown in FIG. 1C. To perform a fast and efficient screen for tight binder small molecules, the small molecular microarray (SMM) technology of the Broad Institute was utilized (FIG. 1C). The SMM is composed of a glass slide with thousands of immobilized small molecules on its surface. The small molecules are organized in a series of spots with their own address on the glass slide. The slides are treated with the GST fused proteins of interest. After stringent washing, the glass surface is then treated with a fluorophore fused GST antibody. Tight binding between the immobilized small molecules on the glass surface and the GST fused proteins will be detectable due to the fluorescence of the antibody. The slides were then deconvoluted to eliminate background binding and subtract GST-binding molecules to produce reliable tight binding hit molecules.

FIG. 1D shows a histogram of replicate averaged hits with background correction for GST-CAD (right) and GST fused intracellular domains of Orai (Left). Cyan colored columns represent hit molecules. FIG. 1 E shows X-Y scatter plots of normalized Z scores for the binding of the chemicals to the fused proteins of interest. Z-scoreA, Z-scoreB and Z scoreC represent three different replicates treated with each protein of interest. Red dots represent background binding. Blue dots are true test compounds and dots in cyan were chosen as potential hits.

The described binding assay was performed three times and a composite Z score was determined for each small molecule based on affinity and consistency (FIG. 1D). 120 small molecules were selected from the large scale assay to be followed up. To save time and resources, the chemical structures and available biological characteristics of these small molecules were prioritized and 51 compounds selected to move forward with secondary screens.

### Identification and Validation of Potential Inhibitors of SOC by NFAT-luc Assays

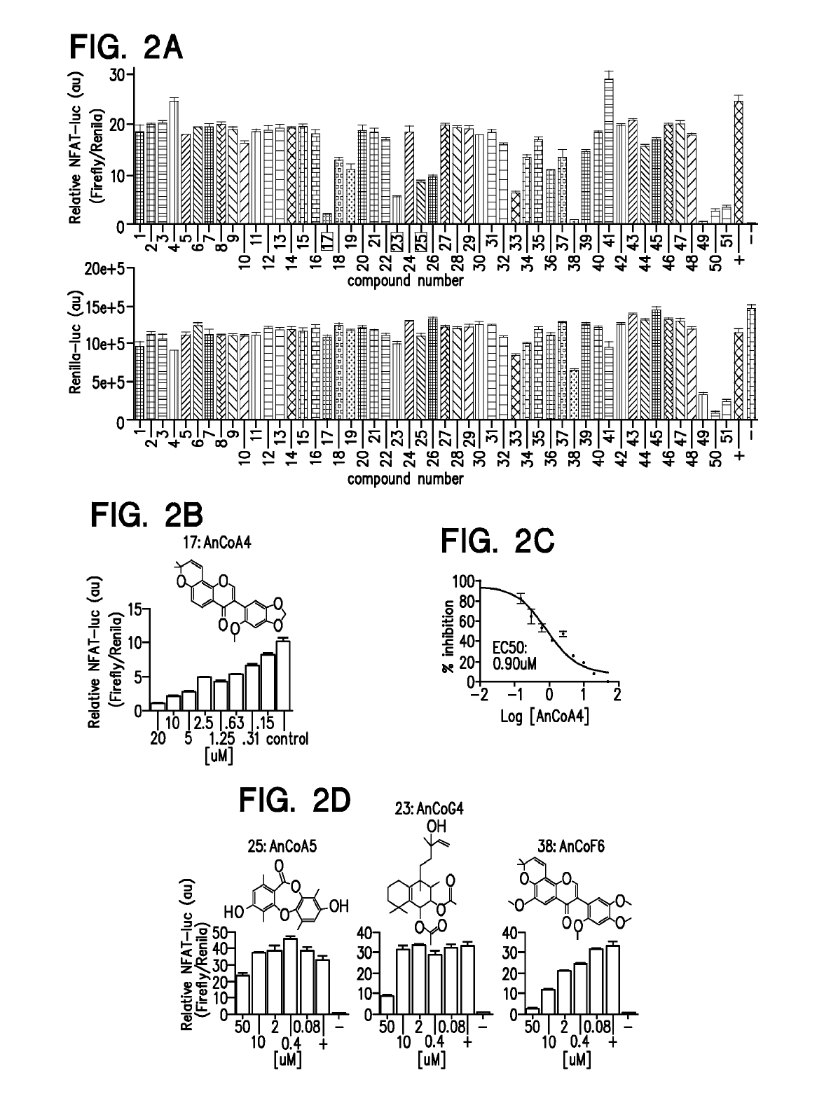

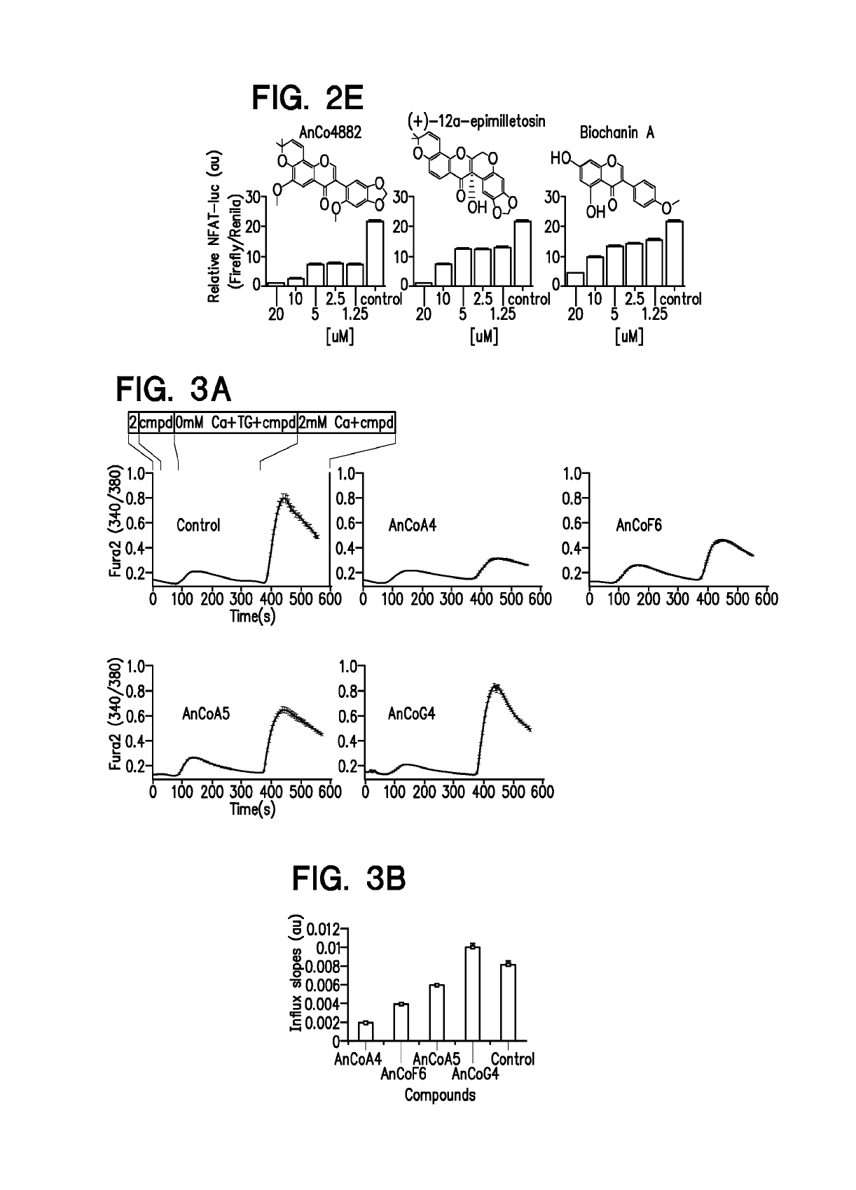

To evaluate the ability of these compounds to inhibit SOC, a NFAT coupled luciferase assay was utilized as a functional secondary screen (FIG. 2A). This assay allows the monitoring of the IL2 promoter dependent production of luciferase that is directly activated by SOC through dephosphorylation of NFAT in T lymphocytes. This assay requires treatment of PMA, a PKC agonist, in parallel with SOC activation. Jurkat T cells were pretreated with each of the 51 compounds for 30 minutes and then activated by treatment of thapsigargin (TG) and PMA. TG depletes the ER stores by inhibiting the SERCA pumps that are responsible for refilling these stores with calcium. This process in turn activates SOC currents.

The result of the screen for functional inhibition of SOC are shown in FIG. 2A. Fifty one compounds identified in the binding assay were tested by NFAT-luc assay to evaluated their SOC inhibitory properties (top). Renilla-luc was also monitored as an internal control of viability of the cells (bottom). Compounds in red rectangles exhibited significant SOC inhibition with normal viability and were chosen for follow up assays.

To further examine the hits produced from the secondary luciferase screen, four compounds AnCoA4, AnCoA5, AnCoG4, and AnCoF6 were selected (FIGS. 2B, C, D). The chemical structure and NFAT-luc assay for serial concentrations of AnCoA4 is shown in FIG. 2B. The Dose response curve calculated from FIG. 2B is shown in FIG. 2C with a calculated EC₅₀ equal to 0.9 μM. FIG. 2D shows the chemical structure and NFAT-luc assay results at different concentrations for selected hit molecules from the SMM screen.

AnCoA4 and AnCoF6 have similar structures with a difference of only one methoxy group. To examine the structure activity relationship of AnCoA4 like molecules, a series of analogs were identified and tested for their ability to inhibit SOC. The chemical structure and NFAT-luc assay results of selected analogs at different concentrations are shown in FIG. 2E. Most of the AnCoA4 analogs showed some degree of inhibition in the low micromolar range by luciferase assay.

### Hits and Analogs Inhibit SOC by Fura2-AM Calcium Imaging in HEK293 Cells

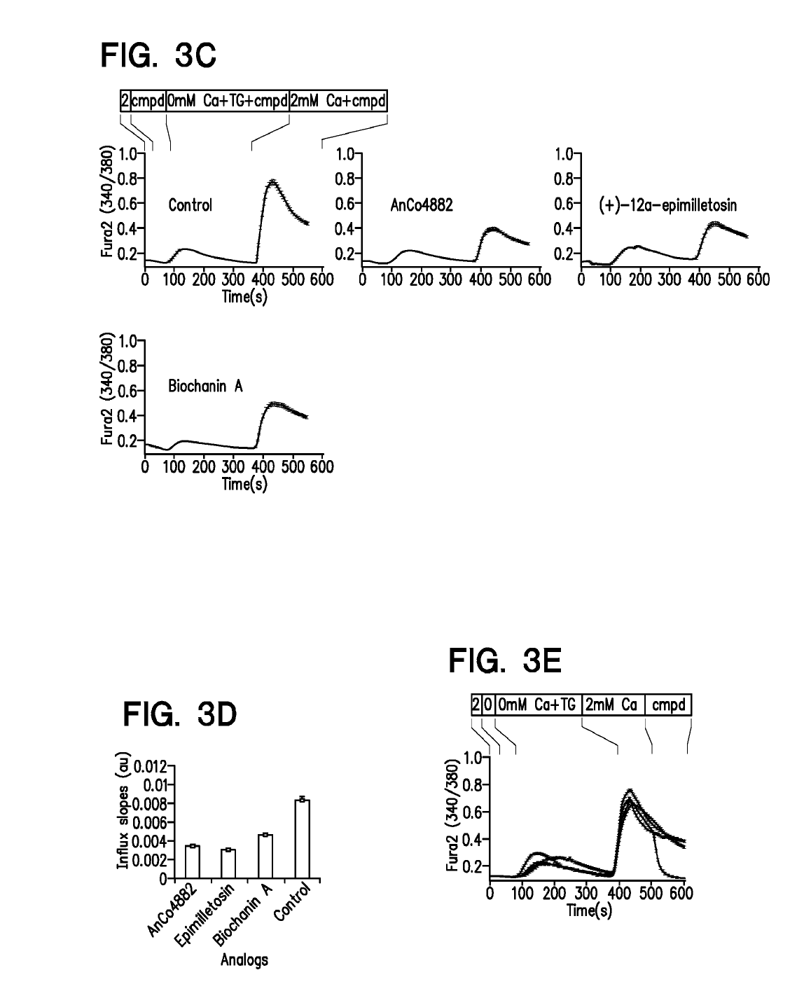

Calcium imaging was utilized for further characterization of the compounds and analogs. Using Fura2-AM, a known ratiometric calcium indicator, SOC dependent intracellular calcium rise and lack thereof was measured after treatment of our compounds (FIG. 3A-B). FIG. 3A shows the results of treating cells with DMSO or 50 uM concentrations of hit molecules from the SMM screen. 1 minute before treatment of Thapsigargin. FIG. 3B shows a bar graph of the slopes of calcium influx from fura2-AM imaging for hit molecules from the SMM screen.

Pretreatment of cells by these compounds and their analogs, e.g., AnCoA4, significantly reduced SOC dependent intracellular calcium influx (FIG. 3C-D). FIG. 3C shows the results of treating cells with DMSO or 50uM concentrations of the commercially available analogs. FIG. 3D shows a bar graph of the slopes of calcium influx from fura2-AM imaging for the commercially available analogs. This observation was in line with the previous luciferase assays and confirmed the ability of these compounds to inhibit SOC.

Although potent inhibitors of SOC, these compounds showed no inhibition when applied after ER store depletion. LaCl, a general blocker of divalent channels, was also used as a comparison for the post treatment experiment. FIG. 3E shows the results of post SOC activation treatment of the compounds. HEK293 cells were treated with DMSO (black), LaCl at 1 uM (red), AnCoA4 at 50 uM (orange), and BiochaninA at 50 uM (green) at minute 510 of the calcium imaging. The intracellular calcium levels quickly drop after LaCl treatment whereas the compound treatment had no effect on the calcium levels.imaging.

### T and B Lymphocyte Activating Genes are Down-Regulated by RT PCR After Treatment of AnCoA4

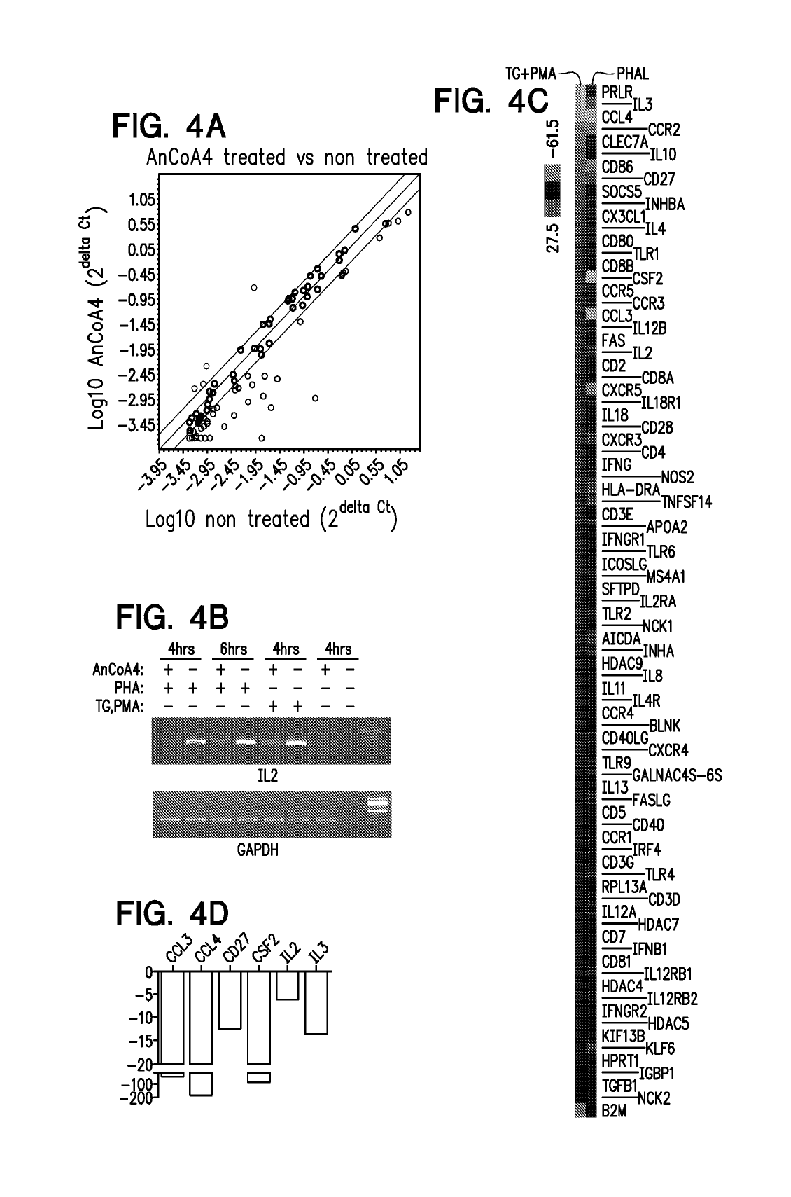

The ability of AnCoA4 to inhibit activation of jurkat T lymphocytes was investigated (FIG. 4A). As IL2 gene production was previously investigated indirectly using a luciferase assay, the production of 83 more genes was monitored using real time quantitative PCR (qRT PCR). FIG. 4A shows the log scaled fold downregulation of 84 genes by qRT PCR. Jurkat T cells were treated with DMSO and AnCoA4 30 minutes before activation with PHA. Total RNA was extracted after 4 hours. Green dots represent genes down-regulated by at least two folds. Red dots represent genes up-regulated by at least 2 folds. FIG. 4B shows semi qRT PCR of IL2 expression with and without treatment of AnCoA4 in jurkat T cells. Cells were pretreated with AnCoA4 30 minutes before activation with PHA or thapsigargin plus PMA for various time length of activation.

These genes are known to be up regulated significantly after activation of the human T and B lymphocytes. Here, jurkat T lymphocytes were pretreated with 50 uM of AnCoA4 for 30 minutes following by treatment of phytohaemagglutinin (PHAL) for activation of the lymphocytes (see FIG. 4B). mRNA levels for most genes were significantly lower in the AnCoA4 treated cells in comparison with the control sample. IL2, a hallmark of T lymphocyte activation was down regulated about 6 folds and other related genes like CCL4, a chemoattractant for mainly natural killer cells, showed a down regulation close to 180 folds (FIGS. 4C-D). FIG. 4C shows a heat map representation of the expression of 84 genes. FIG. 4D shows a bar graph representation of the significantly down-regulated genes from qRT PCR experiment. The ability of the AnCoA4 alone for independent up regulation of IL2 gene was also tested and no effects were observed.

### Abbreviations

Store-operated calcium channels (SOCs); Small Molecule Microarray (SMM); phospholipase C (PLC); inositol 1,4,5-trisphosphate (IP3); endoplasmic reticulum (ER); calcium release-activated calcium channel (CRAC); plasma membrane (PM); Ca²⁺influx factor (CIF); G-protein coupled receptor (GPCR); CRAC Activating Domain (CAD); plasma membrane (PM); protein kinase C (PKC); phosphatidylinisitol 4,5-bisphosphate (PIP2); Nuclear Factor for Activated T cells (NFAT); Diacylglycerol (DAG); Severe Combined Immunodeficiency (SCID) syndrome.

What is claimed is:

1. A method for modulating a store operated calcium channel in a cell, the method comprising:
contacting the cell with a store operated calcium channel modulating effective amount of a compound selected from the group consisting of a chromenone, an octahydro-naphthalene, a dibenzo-diheteropin-1 1-one, a 3a,7a-dihydro-4H-benzofuran-5-one, a tetrahydro-furo[3,4-c]furan-1-one and a 3,3a,4,5-tetrahydro-2H-benzofuran-6-one.

2. The method of claim 1, wherein the compound is a chromenone of the structure of formula (I): 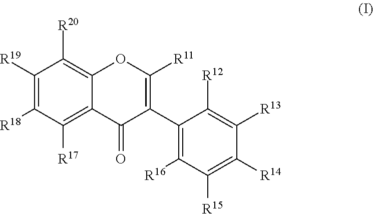
wherein R¹¹, R¹², R¹³, R¹⁴, R¹⁵, R¹⁶, R¹⁷, R¹⁸, R¹⁹ and R²⁰ are independently selected from hydrogen, an aliphatic, an alkoxy, an acyloxy, an aryloxy, an amino, an aryl, a carbonyl, cyano, hydroxyl, a halogen, a heterocycle, a thiol and nitro, wherein optionally two of R¹², R¹³, R¹⁴, R¹⁵, R¹⁶, R¹⁷, R¹⁸, R¹⁹ and R²⁰ can be cyclically linked;

3. The method of claim 1, wherein the compound is a chromenone of the structure of formula (II): 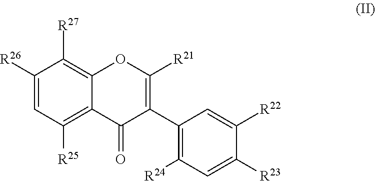
wherein R²¹, R²², R²³, R²⁴, R²⁵, R²⁶ and R²⁷ are independently selected from hydrogen, an aliphatic, an alkoxy, an acyloxy, an aryloxy, an amino, an aryl, a carbonyl, cyano, hydroxyl, a halogen, a heterocycle, a thiol and nitro, wherein optionally two of R²², R²³, R²⁴, R²⁵, R²⁶ and R²⁷ can be cyclically linked.

4. The method of claim 1, wherein the compound is a chromenone of one of the following structures: 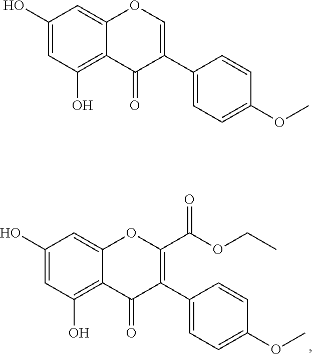 [SMILES: COc1ccc(-c2coc3cc(O)cc(O)c3c2=O)cc1 ; CCOC(=O)c1oc2cc(O)cc(O)c2c(=O)c1-c1ccc(OC)cc1]
or a substituted version thereof.

5. The method of claim 1, wherein the compound is a chromenone of the structure of formula (Ill): 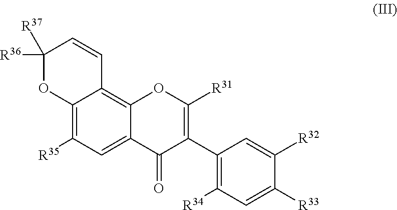
wherein R³¹, R³², R³³, R³⁴ and R³⁵ are independently selected from hydrogen, an aliphatic, an alkoxy, an acyloxy, an aryloxy, an amino, an aryl, a carbonyl, cyano, hydroxyl, a halogen, a heterocycle, a thiol and nitro; and
R³⁶ and R³⁷ are independently selected from hydrogen, an aliphatic, an alkoxy, an acyloxy, an aryloxy, an amino, an aryl, a carbonyl, cyano, hydroxyl, a heterocycle and a thiol.

6. The method of claim 1, wherein the compound is a chromenone of one of the following structures: 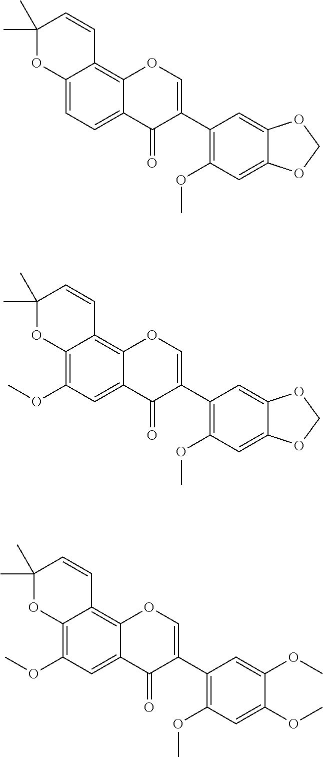
or a substituted version thereof.

7. The method of claim 1, wherein the compound is an octahydro-naphthalene compound of formula (IV): 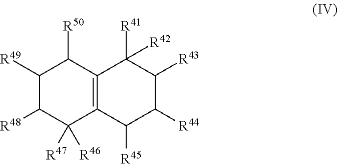
wherein R⁴¹, R⁴², R⁴³, R⁴⁴, R⁴⁵, R⁴⁶, R⁴⁷, R⁴⁸, R⁴⁹ and R⁵⁰ are independently selected from hydrogen, an aliphatic, an alkoxy, an acyloxy, an aryloxy, an amino, an aryl, a carbonyl, cyano, hydroxyl, a heterocycle and a thiol.

8. The method of claim 1, wherein the compound is an octahydro-naphthalene compound of formula (V): 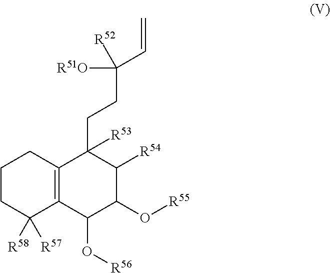
wherein R⁵¹, R⁵², R⁵³, R⁵⁴, R⁵⁵, R⁵⁶, R⁵⁷ and R⁵⁸ are independently selected from hydrogen, an aliphatic, an alkoxy, an acyloxy, an aryloxy, an amino, an aryl, a carbonyl, cyano, hydroxyl, a heterocycle and a thiol. In particular embodiments, in formula (V), R⁵¹, R⁵⁵ and R⁵⁶ are independently selected

9. The method of claim 1, wherein the compound is an octahydro-naphthalene compound of the following structure: 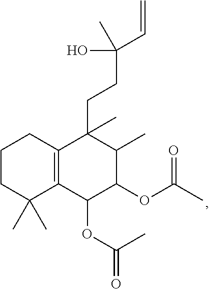 [SMILES: C=CC(C)(O)CCC1(C)C2=C(C(OC(C)=O)C(OC(C)=O)C1C)C(C)(C)CCC2]
or a substituted version thereof.

10. The method of claim 1, wherein the compound is a dibenzo-diheteropin-11-one compound of formula (VI): 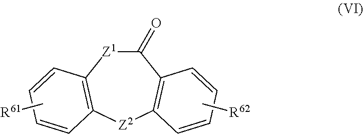
wherein R⁶¹ and R⁶² are independently one or more groups, each R⁶¹ and R⁶² independently selected from hydrogen, an aliphatic, an alkoxy, an acyloxy, an aryloxy, an amino, an aryl, a carbonyl, cyano, hydroxyl, a halogen, a heterocycle, a thiol and nitro;
Z¹ and Z² are independently selected from O, S and NR, where R is hydrogen or alkyl.

11. The method of claim 1, wherein the compound is a dibenzo-diheteropin-11-one compound of formula (VII): 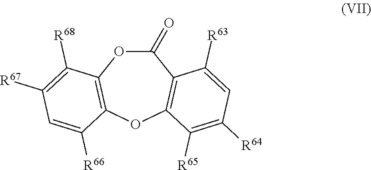
wherein R⁶³, R⁶⁴, R⁶⁵, R⁶⁶, R^{67l and R}⁶⁸ are independently selected from hydrogen, an aliphatic, an alkoxy, an acyloxy, an aryloxy, an amino, an aryl, a carbonyl, cyano, hydroxyl, a halogen, a heterocycle, a thiol and nitro.

12. The method of claim 1, wherein the compound is a dibenzo-diheteropin-11-one compound of the following structure: 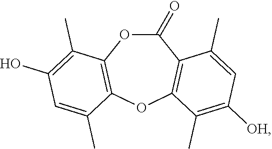 [SMILES: Cc1cc(O)c(C)c2c1Oc1c(C)c(O)cc(C)c1C(=O)O2]
or a substituted version thereof.

13. The method of claim 1, wherein the compound is a dihydro-benzofuran-5-one compound of formula (VIII): 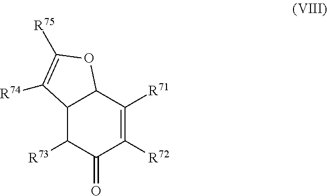
wherein R⁷¹, R⁷², R⁷³, R⁷⁴ and R⁷⁵ are independently selected from hydrogen, an aliphatic, an alkoxy, an acyloxy, an aryloxy, an amino, an aryl, a carbonyl, cyano, hydroxyl, a halogen, a heterocycle, a thiol and nitro, wherein optionally two adjacent groups of R⁷¹, R⁷², R⁷³, R⁷⁴ and R⁷⁵ can be cyclically linked.

14. The method of claim 1, wherein the compound is a dihydro-benzofuran-5-one compound of formula (IX): 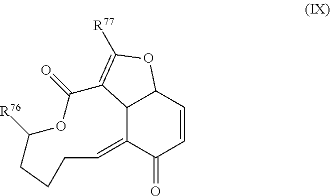
wherein R⁷⁶ and R⁷⁷ are independently selected from hydrogen, an aliphatic, an alkoxy, an acyloxy, an aryloxy, an amino, an aryl, a carbonyl, cyano, hydroxyl, a heterocycle and a thiol.

15. The method of claim 1, wherein the compound is a dihydro-benzofuran-5-one compound of the following structure: 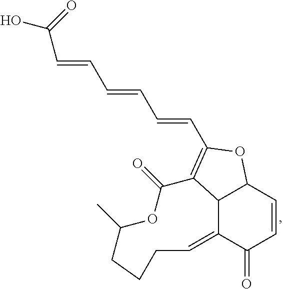 [SMILES: CC1CCC/C=C2/C(=O)C=CC3OC(/C=C/C=C/C=C/C(=O)O)=C(C(=O)O1)C23]
or a substituted version thereof.

16. The method of claim 1, wherein the compound is a tetrahydrofuro-furan-1-one compound of formula (X): 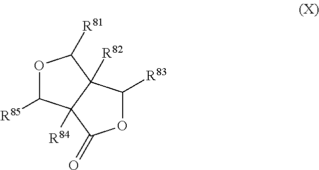
wherein R⁸¹, R⁸², R⁸³, R⁸⁴ and R⁸⁵ are independently selected from hydrogen, an aliphatic, an alkoxy, an acyloxy, an aryloxy, an amino, an aryl, a carbonyl, cyano, hydroxyl, a heterocycle and a thiol.

17. The method of claim 1, wherein the compound is a tetrahydrofuro-furan-1-one compound of formula (XI): 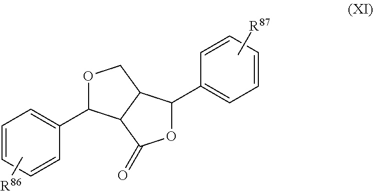
wherein R⁸⁶ and R⁸⁷ are independently one or groups, each R⁸⁶ and R⁸⁷ independently selected from hydrogen, an aliphatic, an alkoxy, an acyloxy, an aryloxy, an amino, an aryl, a carbonyl, cyano, hydroxyl, a halogen, a heterocycle, a nitro and a thiol.

18. The method of claim 1, wherein the compound is a tetrahydrofuro-furan-1-one compound of the following structure: 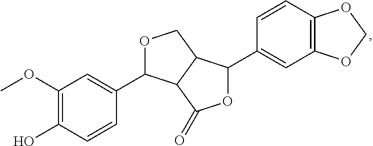 [SMILES: COc1cc(C2OCC3C(c4ccc5c(c4)OCO5)OC(=O)C23)ccc1O]
or a substituted version thereof.

19. The method of claim 1, wherein the compound is a tetrahydro-benzofuran-6-one compound of formula (XII): 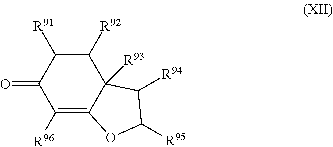
wherein R⁹¹, R⁹², R⁹³, R⁹⁴, R⁹⁵ and R⁹⁶ are independently selected from hydrogen, an aliphatic, an alkoxy, an acyloxy, an aryloxy, an amino, an aryl, a carbonyl, cyano, hydroxyl, a heterocycle and a thiol.

20. The method of claim 1, wherein the compound is a tetrahydro-benzofuran-6-one compound of formula (XIII): 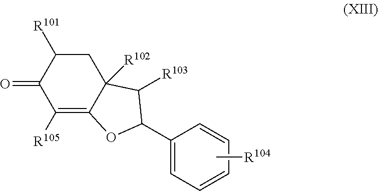
wherein R¹⁰¹, R¹⁰², R¹⁰³ and R¹⁰⁵ are independently selected from hydrogen, an aliphatic, an alkoxy, an acyloxy, an aryloxy, an amino, an aryl, a carbonyl, cyano, hydroxyl, a heterocycle and a thiol;
R¹⁰⁴ is one or groups, each R¹⁰⁴ independently selected from hydrogen, an aliphatic, an alkoxy, an acyloxy, an aryloxy, an amino, an aryl, a carbonyl, cyano, a halogen, hydroxyl, a heterocycle, nitro and a thiol, wherein optionally two R¹⁰⁴ groups can be cyclically linked.

21. The method of claim 1, wherein the compound is a tetrahydro-benzofuran-6-one compound of the following structure: 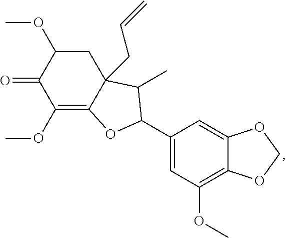 [SMILES: C=CCC12CC(OC)C(=O)C(OC)=C1OC(c1cc(OC)c3c(c1)OCO3)C2C]
or a substituted version thereof.

22. A pharmaceutical composition comprising a store operated calcium channel modulating compound selected from the group consisting of a chromenone, an octahydro-naphthalene, a dibenzo-diheteropin-11-one, a dihydro-benzofuran-5-one, a tetrahydro-furofuran-1-one and a tetrahydro-benzofuran-6-one.
